(19) United States
(12) Patent Application Publication
(10) Pub. No.: US 2024/0285263 A1
(43) Pub. Date: Aug. 29, 2024

(54) SAMPLE COLLECTION DEVICE AND SYSTEM

(71) Applicant: 3M INNOVATIVE PROPERTIES COMPANY, St. Paul, MN (US)

(72) Inventors: Michael R. Berrigan, Oakdale, MN (US); Alan R. Dombrowski, Woodbury, MN (US); Laura R. Nereng, Woodbury, MN (US); Audrey A. Sherman, Woodbury, MN (US); Brett J. Sitter, Marine on St. Croix, MN (US); Narina Y. Stepanova, Woodbury, MN (US)

(21) Appl. No.: 18/564,514

(22) PCT Filed: May 24, 2022
(86) PCT No.: PCT/IB2022/054852

§ 371 Date: Nov. 27, 2023

Related U.S. Application Data
(60) Provisional application No. 63/202,143, filed on May 28, 2021.
(60) Provisional application No. 63/227,608, filed on Jul. 30, 2021.

(51) Int. Cl. A61B 10/00; G01N 33/543

(52) U.S. Cl. CPC A61B 10/0051; G01N 33/54388; G01N 33/54391; A61B 2010/0087

(57) ABSTRACT

A sample collection system includes a housing comprising a first part and a second part, the first and second parts being movable relative to one another; porous sample collection media disposed along an airflow path in the first part; and an assay disposed on the second part and constructed to receive a sample captured by the porous sample collection media. The first and second parts are rotatably movable relative to one another. Moving the first and second parts relative to one another aligns the porous sample collection media with at least a portion of the assay. The assay may be a lateral flow assay or a vertical flow assay. The media is constructed to collect a sample from exhalation airflow. A collected sample may be eluted from the media onto the assay using a liquid.

### FIELD

The present disclosure relates to a sample collection device and system. The present disclosure relates to a bioaerosol collection device and system.

### BACKGROUND

Diagnostic tests used to test for the presence of a virus or other pathogen in the airways, throat, or nasopharynx typically involve the insertion of a swab into the back of the nasal passage, the mid-turbinate area of the nasal passage, the anterior nares, or the throat to obtain a sample. The swab is then inserted into a container and analyzed or sent to a lab for processing. Other diagnostic tests involve collecting a saliva sample and then placing it in a container. Currently available at-home viral tests (e.g., COVID-19 tests) involve a nasal swab and a test kit (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

There is a need for an inexpensive, simple to use, and reliable sample collection system that may be used by laypeople for testing for the presence of a target virus, target pathogen, or other target analyte, in a collected sample. The sample collection system may include a sample collection device for collecting a sample from exhalation airflow and a testing assay to determine the presence or absence of the target virus, target pathogen, or other target analyte, in the collected sample.

It is desirable to provide a system that includes both a sample collector device and rapid antigen testing in an integrated system. The integrated system may advantageously be self-contained and optionally sterile. A self-contained and sterile system may improve accuracy and reliability of pathogen testing due to the reduced contamination and background noise, unlike swabs and other test collection devices which may be contaminated upon use and/or during testing.

It is further desirable to provide a system which, after sample collection and optional testing, remains closed and self-contained to contain any potential virus or pathogen, and which may be safely disposed of among ordinary waste collection.

According to an embodiment, a sample collection system includes a housing comprising a first part and a second part, the first and second parts being movable relative to one another; porous sample collection media disposed along an airflow path in the first part: and an assay disposed on the second part and constructed to receive a sample captured by the porous sample collection media. The airflow path defines a through opening in the first part and the second part, wherein the porous sample collection media occludes the through opening of the first part. The first part may include a screen disposed in the airflow path in front of the porous sample collection media.

The first and second parts are rotatably movable relative to one another. The first part may form an outer tube and the second part may form an inner tube at least partially disposed inside the outer tube. The first part and the second part have a first position that is a sample collection position and a second position that is a testing position. In the first position, through holes extending through the first part and the second part are aligned. In the second position, the porous sample collection media is aligned with the assay.

The first part may further include a mouthpiece aligned with the porous sample collection media. The mouthpiece may be removably coupled with the first part.

The porous sample collection media is constructed to capture a sample of viruses, pathogens, or other analytes from exhalation airflow. The porous sample collection media may be made of nonwoven material. The nonwoven material may include polylactic acid, polypropylene, or a combination thereof. The nonwoven material may carry an electrostatic charge.

The assay may be a lateral flow assay or a vertical flow assay. The assay is constructed to determine the presence or absence of a target virus, pathogen, or analyte in the collected sample.

The assay may define a strip of material with a length, and wherein the length is parallel to a longitudinal axis of the second part. The assay may define a strip of material with a length, and wherein the length is transverse to a longitudinal axis of the second part.

According to an embodiment, a kit includes the sample collection system and instructions for collecting a sample and testing the sample using the assay. The instructions may include instructions to: exhale along the airflow path to capture a sample in the porous sample collection media: move the first and second parts relative to one another to align the porous sample collection media with the assay: apply a liquid to the porous sample collection media: and read a result in a result display of the assay.

### BRIEF DESCRIPTION OF FIGURES

FIGS. 1A and 1B are perspective views of a sample collection system according to an embodiment.

FIG. 1C is a cross-sectional side view of the sample collection system of FIG. 1A.

FIGS. 2A and 2B are perspective views of a first part of the housing of the sample collection system of FIG. 1A.

FIG. 2C is a partial cut-off perspective view of the first part of the housing of FIG. 2A.

FIG. 3A is a perspective view of a second part of the housing of the sample collection system of FIG. 1A.

FIG. 3B is a partial cut-off perspective view of the second part of the housing of FIG. 3A.

FIGS. 4A and 4B are schematic cross-sectional views of the sample collection system of FIG. 1A.

FIGS. 4C and 4D are schematic cross-sectional views of a sample collection system with a built-in liquid reservoir according to an embodiment.

FIG. 5A is a perspective view of the sample collection system of FIG. 1A in a testing position.

FIG. 5B is a partial cut-off perspective view of the sample collection system of FIG. 5A.

FIGS. 6A and 6B are perspective views of a sample collection system according to an alternative embodiment.

FIG. 6C is a cross-sectional side view of the sample collection system of FIG. 6A.

FIGS. 7A and 7B are perspective views of a first part of the housing of the sample collection system of FIG. 6A.

FIG. 7C is a partial cut-off perspective view of the first part of the housing of FIG. 7A.

FIG. 8A is a perspective view of the sample collection system of FIG. 6A in a testing position.

FIG. 8B is a partial cut-off perspective view of the sample collection system of FIG. 8A.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The terms “downstream” and “upstream” refer to a relative position based on a direction of exhalation airflow through the device. For example, the upstream-most element of the device is the mouthpiece element, and the downstream-most element of the device is the exhalation outlet element.

All headings provided herein are for the convenience of the reader and should not be used to limit the meaning of any text that follows the heading, unless so specified.

The term “i.e.” is used here as an abbreviation for the Latin phrase id est, and means “that is,” while “e.g.” is used as an abbreviation for the Latin phrase exempli gratia and means “for example.”

The term “not substantially” as used here has the same meaning as “not significantly,” and can be understood to have the inverse meaning of “substantially,” i.e., modifying the term that follows by not more than 25%, not more than 10%, not more than 5%, or not more than 2%.

All scientific and technical terms used herein have meanings commonly used in the art unless otherwise specified. The definitions provided herein are to facilitate understanding of certain terms used frequently herein and are not meant to limit the scope of the present disclosure.

The term “about” is used here in conjunction with numeric values to include normal variations in measurements as expected by persons skilled in the art and is understood have the same meaning as “approximately” and to cover a typical margin of error, such as ±5% of the stated value. Moreover, unless otherwise indicated, all numbers expressing quantities, and all terms expressing direction/orientation (e.g., vertical, horizontal, parallel, perpendicular, etc.) in the specification and claims are to be understood as being modified in all instances by the term “about.”

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc, or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to a sample collection device and system. The present disclosure relates to a bioaerosol sample collection device. The present disclosure further relates to a system that includes both sample collection and testing capabilities.

The sample collection system includes a sample collection device with porous sample collection media along an airflow path defined by the device housing. The porous sample collection media is constructed to capture viruses, pathogens, or other analytes, carried in an exhalation airflow. The system may further include a sample testing assay. A liquid may be passed through the porous sample collection media to elute the sample, including pathogens, viruses, or other analytes, bound to the porous sample collection media, forming an eluent. The eluent may then be analyzed using the assay. The eluent may be directed from the sample collection media to the assay. The liquid may be provided as a metered dose of liquid housed in a liquid reservoir. The liquid may alternatively be applied from a separate applicator. The device or container including the liquid could have a tamper-indicating portion that indicates if the liquid has been tampered with so that the user could see that before use. An exemplary tamper-indicating device or container is one that has a foil or similar cover. If the cover has been pierced or removed, that would be evident to the user. Other tamper-indicating features include the delivery of the liquid resulting in breakage or damage to the liquid container such that the user could see that it had been used or tampered with, colored dye indicating user or tampering, or breakpoints (e.g. perforations, thin walls, torque breaking plastic portions, and/or spring like designs) that indicate to the user if the liquid containing device has been used or tampered with.

According to an embodiment, the sample collection device includes a housing. The housing includes a first part and a second part. The first and second parts are movable relative to one another. The porous sample collection media may be disposed along an airflow path in the first part. The assay may be disposed on (e.g., attached to) the second part. The porous sample collection media is constructed to capture a sample from an exhalation of a user. The assay is constructed to receive the sample captured by the porous sample collection media.

In some embodiments, the assay may be a separate element from the sample collection device. The assay may be configured to attach to (e.g., slide or snap in) the sample collection device. The sample collection device may include a receptable for receiving the assay. The assay may be a replacement element with the sample collection device. The assay may be integral with the sample collection device. The assay may form a unitary element with the housing of the sample collection device.

The housing may define one or more openings that form the airflow path. In some cases. the housing includes at least two openings that form the airflow path. The two openings may be through holes in a structure (e.g., the first part and the second part) of the housing. According to an embodiment, the housing includes a first part and a second part, and two through openings in each of the first and second part defining the airflow path. One opening may be an air inlet and another opening may be an air outlet to allow through flow. The porous sample collection media may be disposed or fixed along the airflow path. For example, the porous sample collection media may be fixed or attached to the housing such that the porous sample collection media covers at least one of the openings (e.g., the inlet opening) forming the airflow path. The porous sample collection media may occlude a through opening in the first part.

The housing may include a liquid inlet constructed to receive a liquid for eluting the sample. The liquid inlet may be constructed to direct the liquid onto the porous sample collection media. In some embodiments, the housing includes a liquid reservoir containing a metered dose of liquid. Alternatively, the liquid may be provided separately, e.g., in a dropper or other applicator. The liquid reservoir containing the metered dose of liquid may be deformable and configured to discharge fluid from the metered fluid dose element upon application of pressure to the liquid reservoir. In some embodiments the liquid reservoir is pierceable, frangible, or rupturable.

According to an embodiment, the first and second parts may be slidably movable relative to one another. The first and second parts may be rotatably movable relative to one another. The first and second parts may be slidably and rotatably movable relative to one another. For example.

the first part may form an outer tube and the second part may form an inner tube at least partially disposed inside the outer tube, such that the inner and outer tubes can be rotated relative to one another. The first part and the second part may have a first position that is a sample collection position and a second position that is a testing position. In the first position, the through holes extending through the first part and the second part are aligned (e.g., registered). According to an embodiment, moving the first and second parts to the second position aligns the porous sample collection media with at least a portion of the assay. For example, moving the first and second parts to the second position may align (e.g., register) the porous sample collection media with a sample receiving area of the assay. Moving the first and second parts to the second position may cause the porous sample collection media to come into contact with (e.g., touch) the assay. Moving the first and second parts to the second position may cause the porous sample collection media to come into contact with (e.g., touch) a sample receiving area of the assay.

In embodiments where a metered dose of liquid is provided in a liquid reservoir, the liquid reservoir may be attached to the housing, e.g., to the fluid inlet port. The liquid reservoir may be permanently attached to the fluid inlet port. In some embodiments, the liquid reservoir may be integral with the fluid inlet port. The liquid reservoir may be removably attached to the fluid inlet port. In some embodiments, the liquid reservoir may be a replaceable element onto the fluid inlet port.

The liquid dispensed onto the porous sample collection media may be an aqueous liquid. The liquid may be a buffer solution. The liquid may be an aqueous buffer solution. The liquid may be a saline solution. The liquid may include a surfactant. The liquid may have a contact angle of greater than 90 degrees when measured on the porous sample collection media. The liquid may be a saline solution including a surfactant. The liquid (e.g., a buffer or a saline solution) may include from 0.1 wt-% or more or 0.5 wt-% or more, and up to 1 wt-% or up to 2 wt-% of surfactant. When provided as a metered dose, the liquid may have a volume of 50 μL to 500 μL.

The liquid may be applied onto the loaded porous sample collection media. The liquid may travel through the surface and thickness of the loaded porous sample collection media and flow off of the porous sample collection media carrying any virus, pathogen, or other analyte, that was present on the loaded porous sample collection media. This loaded liquid may then be collected and tested, as described herein.

The housing may include a pressure element (e.g., a protrusion or a spring) constructed to apply pressure to the porous sample collection media or the assay to press the porous sample collection media against the assay. The pressure element may be constructed to cause more eluent to flow from the porous sample collection media to the assay.

The housing may include a mouthpiece. The mouthpiece may be aligned with the inlet opening. The mouthpiece may help a user direct exhalation airflow onto the porous sample collection media. The mouthpiece may be integral with the housing or may be removably coupled with the housing. As used herein, the term “mouthpiece” is not limited to exhalation from the mouth. Instead, the term “mouthpieces” merely refers to a exhalation receipt portion or device that is capable of receiving exhalation by the user from, for example, the user's mouth or nose.

The user may exhale into the sample collection system and load the porous sample collection media with a sample of the exhalation airflow to form a loaded porous sample collection media. For example, the user may exhale through the single opening or air inlet or through a mouthpiece. The housing may be constructed such that by exhaling through the single opening, air inlet, or mouthpiece, the exhalation airflow passes through the porous sample collection media. The porous sample collection media is constructed to capture viruses, other pathogens, or other analytes, from the exhalation airflow. The user may then move the first and second parts of the housing relative to one another to bring the loaded porous sample collection media and the assay in alignment. For example, the user may rotate the inner tube relative to the outer tube. By moving the first and second parts to the second position, the porous sample collection media and the assay may be aligned or may touch one another. The user may then apply a liquid to the loaded porous sample collection media to elute the captured sample onto the assay. The user may test the eluent for the presence of a virus, pathogen, or other analyte using the assay. The testing may take place with the loaded porous sample collection media in place in the sample collection system.

According to an embodiment, the sample collection system forms a singular self-contained unit. Providing a self-contained unit allows for convenient shipping and transportation of the sample collection system and for disposal after use. The self-contained unit may have a compact size and may be conveniently carried in a pocket or purse. The self-contained unit may be safely disposed of after use among ordinary waste disposal.

The assay included in the sample collection system may be any suitable assay. In some embodiments, the assay is a lateral flow assay (“LFA”) or a vertical flow assay (“VFA”). LFAs and VFAs are generally paper-based platforms for the detection and quantification of analytes in complex mixtures, including biological samples such as saliva, urine, etc. LFAs and VFAs are typically easy to use and can be used both by professionals in a health care setting or laboratory as well as by lay persons at home. Typically, a liquid sample is placed on the assay in a sample receiving region and is wicked by capillary flow along the device to a test region. LFAs and VFAs are typically based on antigens or antibodies that are immobilized in the test region and that selectively react with the analyte of interest. The result is typically displayed within 5 to 30 minutes. LFAs and VFAs can be tailored for the testing of a variety of viruses and other pathogens, as well as many other types of analytes. According to an embodiment, the assay used in the sample collection system of the present disclosure is constructed for the detection of a target virus, target pathogen, or other target analyte. According to an embodiment, the assay used in the sample collection system of the present disclosure is constructed for the detection of a target virus. target pathogen, or other target analyte, that may be present in the exhalation air flow of a subject.

According to an embodiment, the porous sample collection media is a nonwoven material carrying an electrostatic charge. The electrostatic charge may enable capturing pathogens, viruses. or other analytes from an exhalation airflow. In some cases, the porous sample collection media may be a hydrophobic nonwoven material. In other cases, the porous sample collection media may be a hydrophilic nonwoven material. The porous sample collection media may be a hydrophobic nonwoven material carrying an electrostatic charge configured to capture pathogens, viruses, or other analytes from an exhalation airflow. The porous sample collection media may be a hydrophilic nonwoven material carrying an electrostatic charge configured to capture pathogens. viruses, or other analytes from an exhalation airflow. The term “hydrophobic” refers to a material having a water contact angle of 90 degrees or greater, or from about 90 degrees to about 170 degrees, or from about 100 degrees to about 150 degrees. The term “hydrophilic” refers to a material having a water contact angle of less than 90 degrees. Water contact angle is measured using ASTM D5727-1997 Standard test method for surface wettability and absorbency of sheeted material using an automated contact angle tester.

The porous sample collection media may be formed of any suitable material that is capable of capturing viruses, pathogens, or other analytes from exhalation airflow and releasing the captured viruses, pathogens, or other analytes upon being contacted with an eluent, such as a saline solution. The porous sample collection media may be formed of polymeric material. The porous sample collection media may be formed of a polyolefin. Examples of suitable polyolefins include polypropylene, polylactic acid, and the like, and a combination thereof. In one embodiment the porous sample collection media is formed of polypropylene. In one embodiment the porous sample collection media is formed of polylactic acid. One illustrative porous sample collection media is commercially available from 3M Company (St. Paul MN. U.S.A.) under the trade designation FILTRETE Smart MPR 1900 Premium Allergen. Bacteria & Virus Air Filter Merv 13.

While the porous sample collection media is illustrated here as defining a substantially planar element, it is understood that the porous sample collection media may define any shape when disposed within the housing and along the airflow path. The porous sample collection media may be an ordinarily planar element that is placed along a curved surface. That is, the flat piece of porous sample collection media may be slightly bent to follow the contour of the first part of the housing.

While the porous sample collection media is illustrated here as defining a planar element. it is understood that the porous sample collection media may define any shape when disposed within the housing and along the airflow path. For example, the sample collection media may be pleated. In some embodiments, the pleat frequency is between about 1 pleat per 0.6 cm of media and about 1 pleat per 2 mm of media. In some embodiments, the pleat height is between about 2 mm and about 4 mm.

The porous sample collection media may have a thickness (orthogonal to the major plane) of 200 μm or greater or 250 μm or greater. The porous sample collection media may have a thickness of 750 μm or less or 1000 μm or less. The porous sample collection media may have a thickness of in a range from 200 μm to 1000 μm, or from 250 μm to 750 μm. The porous sample collection media may have major plane surface area (of one side) of 1 cm2 or greater or 2 cm2 or greater. The porous sample collection media may have major plane surface area of 3 cm2 or less or 4 cm2 or less. The porous sample collection media may have major plane surface area in a range from 1 cm2 to 4 cm2, or 2 cm2 to 3 cm2.

The housing may be formed of a rigid material, such as plastic or a paper-based material such as cardboard or cardstock. In some embodiments, the housing is made of plastic. In some embodiments, at least a portion of the housing is transparent. For example, the housing may include transparent material in an area of a result display of the assay. The housing may include a viewing window (either transparent material or an opening) in the area of the result display. In some cases, the entire housing may be made of a transparent material. The housing may further include a cover or sealing layer constructed to prevent contamination before or after use of the system. The cover or sealing layer may be removable (e.g., may be removed before use). The cover or sealing layer may be closable and/or re-closable (e.g., may be closed after use). The cover or sealing layer may include one or more flaps constructed to close the one or more openings of the housing. In one embodiment, the cover includes a flap constructed to close the open end of the housing.

The housing may include a pre-filter or screen disposed in the airflow path in front (upstream) of the porous sample collection media. For example, the first part of the housing may include a screen disposed in the airflow path in front of the porous sample collection media. The screen may be constructed to catch larger particles (larger than viruses or pathogens) and prevent such particles from reaching the porous sample collection media. The exhalation airflow passes through a thickness of the pre-filter or screen. The pre-filter or screen at least partially occludes the air flow path. In some cases, the pre-filter or screen may have a major plane that is orthogonal to the direction of the exhalation airflow passing through the thickness of the pre-filter or screen. The pre-filter or screen may be a non-woven layer configured to filter out larger particles from the exhalation airflow passing through the pre-filter or screen. In some cases, the pre-filter or screen may be a non-woven layer that does not have an electrostatic charge. In some embodiments, the pre-filter or screen does not capture significant amounts of viral material, pathogen material, or other analyte material, and instead allows them to transmit through the pre-filter or screen. In some embodiments, the pre-filter or screen is made of or includes at least one of a plastic mesh, a woven net, a needle-tacked fibrous web, a knitted mesh, an extruded net, and/or a carded or spunbond coverstock.

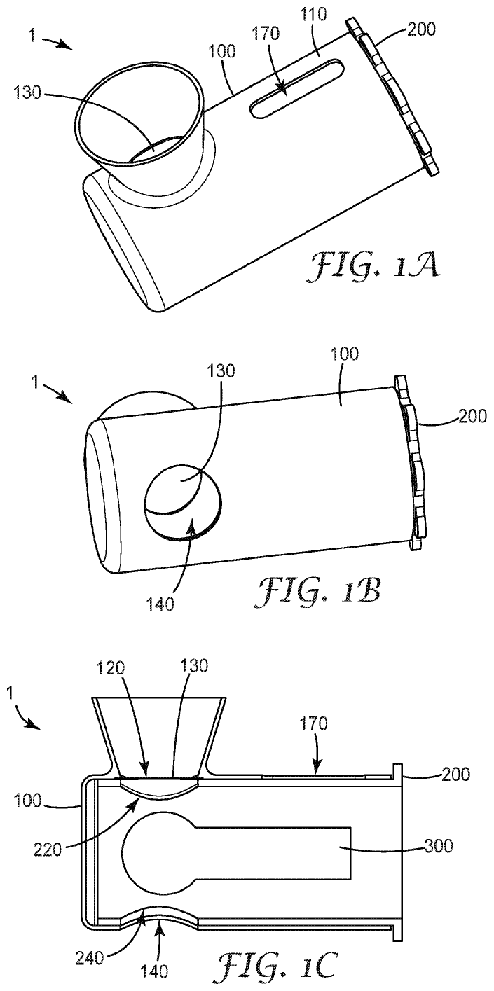

Referring now to FIGS. 1A-1C, a sample collection system 1 with rotatably movable first and second parts 100, 200 is shown. The sample collection system 1 includes a housing 10 made up of a first part 100 and a second part 200. The first and second parts 100, 200 are movable relative to one another. The first part 100 forms an outer tube and the second part 200 forms an inner tube that is at least partially disposed inside the outer tube. The first and second parts 100. 200 are slidably and rotatably movable relative to one another.

An element of porous sample collection media 130 is disposed along an airflow path in the first part 100. An assay 300 is disposed on the second part 200 and constructed to receive a sample captured by the porous sample collection media 130. The airflow path is formed by a through opening 120 in the first part 100 and a through opening 220 in the second part 200. The porous sample collection media 130 occludes the through opening 120 of the first part 100. The airflow path may include a second through opening 140, 240 in each of the first and second parts 100, 200.

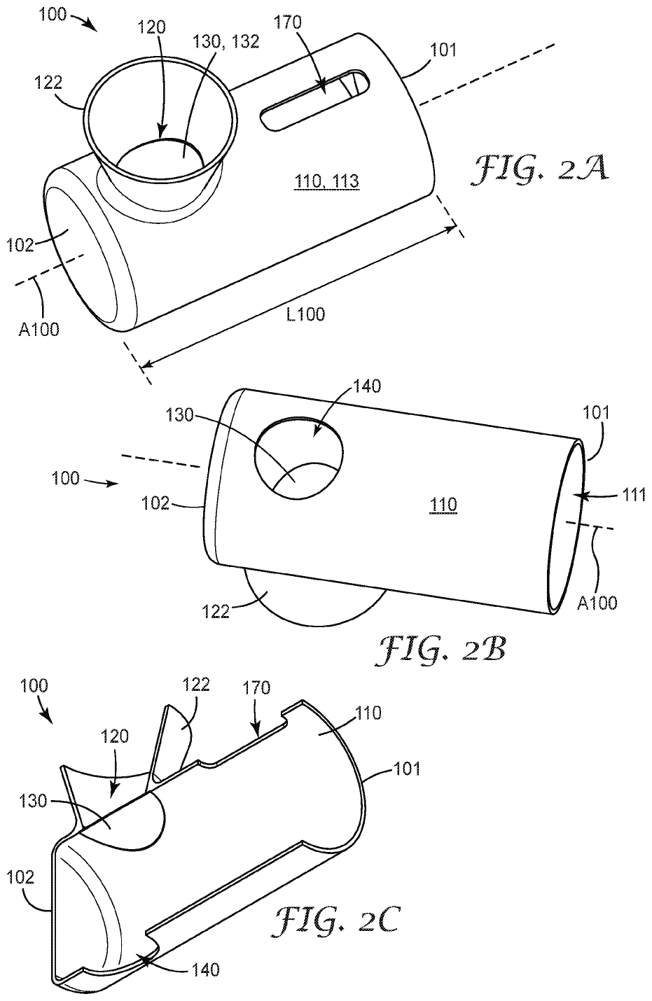

The first part 100 is shown in more detail in FIGS. 2A-2C. The first part 100 has a wall 110 with a first end 101 and a second end 102 and a length L100 extending from the first end 101 to the second end 102. The wall 110 may be cylindrical, as shown, defining a tubular interior 111. Alternatively, the wall 110 may have an outer surface 113 with a non-circular cross section. For example, the outer surface 113 may define protrusions or one or more flat portions that prevent the system 1 from rolling on a table or other surface. The first part 100 may define a longitudinal center axis A100 extending along the length L100.

The first opening 120 extends through the wall 110. The first opening 120 may include a mouthpiece 122. The mouthpiece 122 may be formed by a tubular or frustoconical element, as shown. The mouthpiece 122 may be integral with or may be removably coupled with the wall 110. The mouthpiece 122 may also serve as a liquid inlet port. The first opening 120 is occluded by the porous sample collection media 130. The porous sample collection media 130 may be attached or fixed to the wall 110. The porous sample collection media 130 may be attached or fixed directly to the wall 110 or may be fixed using a support element. In front of (upstream of) the porous sample collection media 130, the first part 100 may include a pre-filter or screen 132. The pre-filter or screen 132 may be provided as a layer of the porous sample collection media 130 on the upstream side.

The first part 100 may also include a second opening 140 as part of the airflow path. The second opening 140 may be disposed opposite of the first through opening 120. Alternatively, the second opening 140 may be disposed anywhere along the wall 100 or may be provided by an open end 150.

The first part 100 may further include a result viewing area 170. The result of the assay

300 test may be viewed through the result viewing area 170. The result viewing area 170 may include transparent material or an opening in the wall 110. In some embodiments, the entire wall 110 is made of transparent material. In some embodiments, the wall 110 includes a window at the result viewing area 170. The result viewing area 170 may be longitudinally aligned (parallel to axis A100) with the first opening 120. The transparent material may be aligned with the assay 300 such that the transparent material overlays the result display 370.

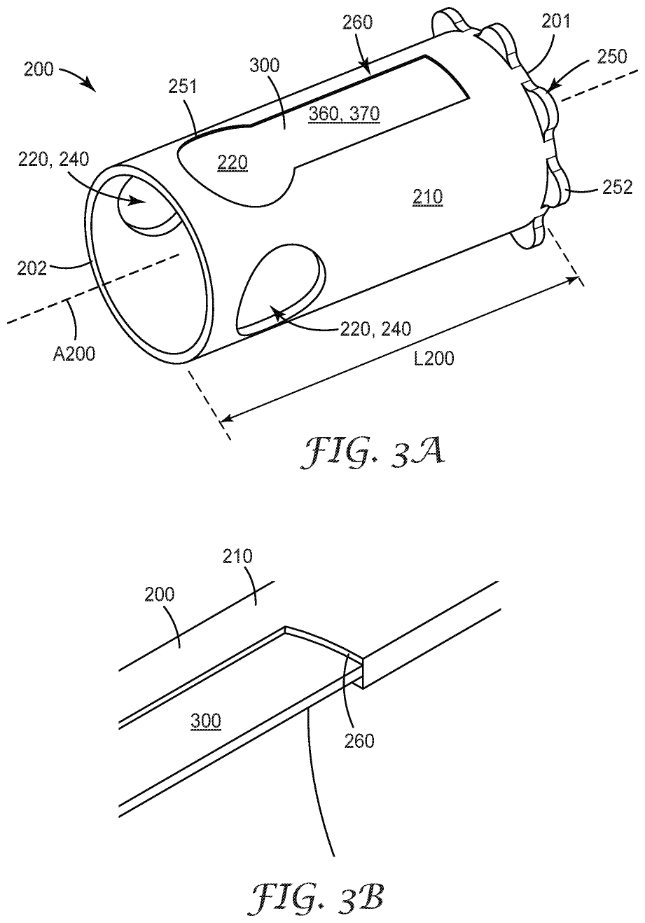

The second part 200 is shown in FIGS. 3A and 3B. The second part 200 has a wall 210 with a first end 201 and a second end 202 and a length L200 extending from the first end 201 to the second end 202. The wall 210 may be cylindrical, as shown, defining a tubular interior 211. The second part 200 may define a longitudinal center axis A200 extending along the length L200.

The first opening 220 extends through the wall 210. The first opening 220 may be aligned with the first opening 120 and mouthpiece 112 of the first part 100. The second part 200 may also include a second opening 240 as part of the airflow path. The second opening 240 may be disposed opposite of the first through opening 220. Alternatively, the second opening 240 may be disposed anywhere along the wall 210 or may be provided by an open end 250. The second opening 240 may be aligned with the second opening 140 of the first part 100.

The second part 200 may include an assay receptacle 260 or well for receiving an assay 300. The assay receptacle 260 may extend along the length L200 of the second part 200, from a sample receiving area 251 toward the first end 201. The sample receiving area 251 may be aligned with the first opening 120 and the porous sample collection media 130 of the first part 100.

The assay 300 is disposed on the second part 200 or within the assay receptacle 260. Any suitable assay may be used, such as a lateral flow assay or vertical flow assay. The assay 300 includes a sample receiving area 330 at or near the first end 301 of the assay and a test area 360 and result display 370 at or near the second end 302 of the assay. The assay 300 has a length L300. The length L300 may be disposed parallel to the length L200 of the second part 200.

The second part 200 may be at least partially disposed within the first part 100 (as shown, for example, in FIGS. 1A-1C). The longitudinal axis A200 of the second part 200 may be coaxial with the longitudinal axis A100 of the first part 100. The second part 200 is preferably rotatable within the first part 100.

The second part 200 may include a grip element 252 to aid in rotating the second part 200 within the first part 100. The grip element 252 may extend from the second end 202 and may extend past the second end 102 of the first part 100 when the second part 200 is disposed inside the first part 100.

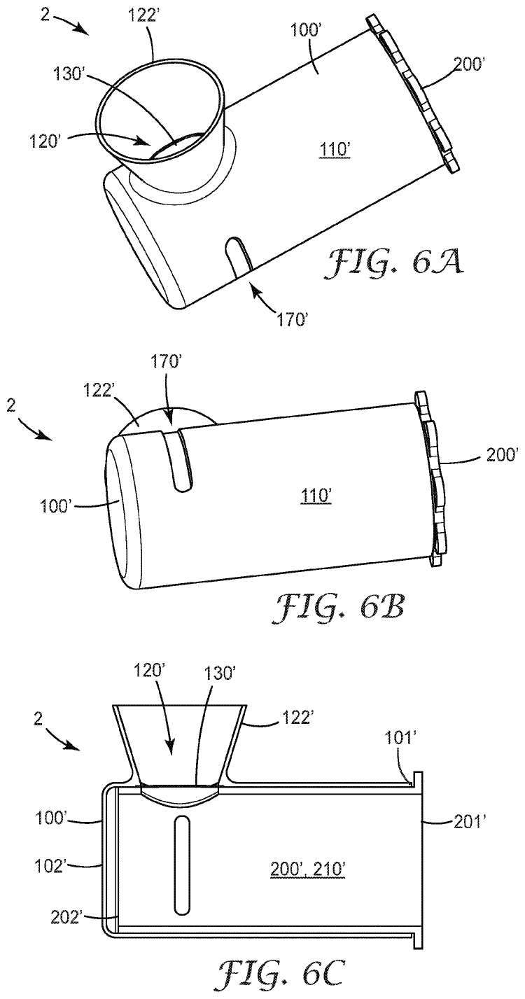

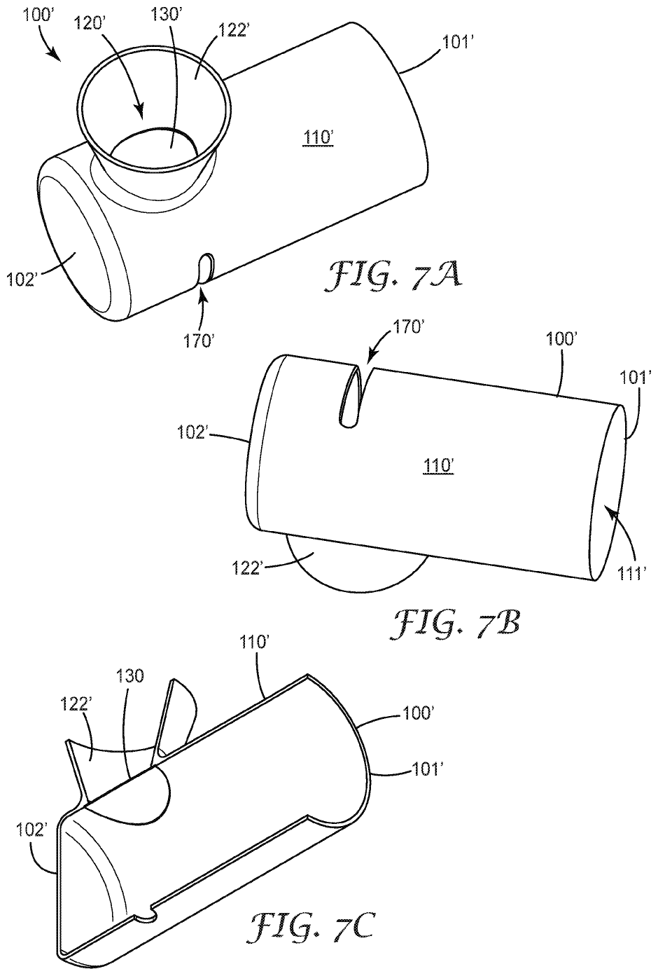

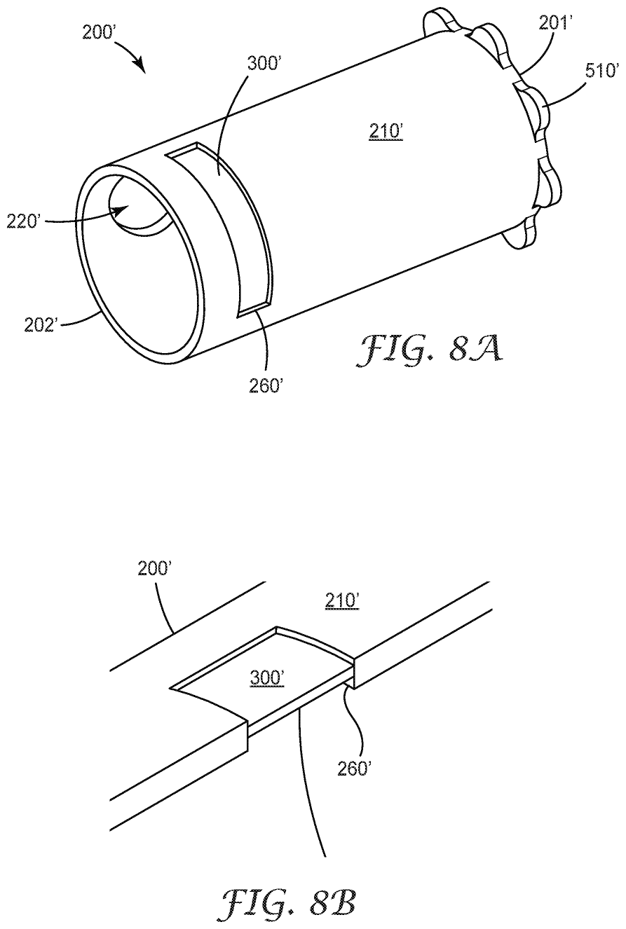

In an alternative embodiment of the system 2, shown in FIGS. 6A-8B, the assay 300′ is aligned transverse to the longitudinal axis A200 of the second part 200′. The system 2 is otherwise similar to the system 1 shown in FIGS. 1A-1C except that the various features are configured to accommodate the transverse assay 300′. The assay receptacle 251′ is disposed along the wall 210′ of the second part 200′ transverse to the longitudinal axis A200. The assay 300′ has a length L300′ that is disposed transverse to the axis A200 of the second part 200. The assay receptacle 251′ and assay 300 may be axially aligned with the first openings 120, 220. The result viewing area 170′ of the first part 100 is also aligned axially with the first opening 120. To accommodate the transverse assay 300′, the second openings 140′, 240′ may be placed elsewhere along the housing, not aligned with the first openings 120, 220 and assay 300′. The second openings 140′, 240′ may be provided by the open end 150′, 250′.

In the transverse assay configuration, the assay 300′ follows the contour of the wall 210′. Any suitable assay may be used, such as a lateral flow assay or vertical flow assay. Lateral flow assays commonly have a layered constructions. Some of the layers may not extend the entire length of the assay. It may be beneficial to orient the assay 300′ such that the curvature does not cause delamination of the various layers of the assay 300′ but rather helps push the layers together. This may be achieved by orienting any shorter (less than full length L300′ of the assay 300′) layers to toward the interior 211′ of the second part 200′. Orienting the assay 300′ in this way may improve the performance of the sample collecting system 2.

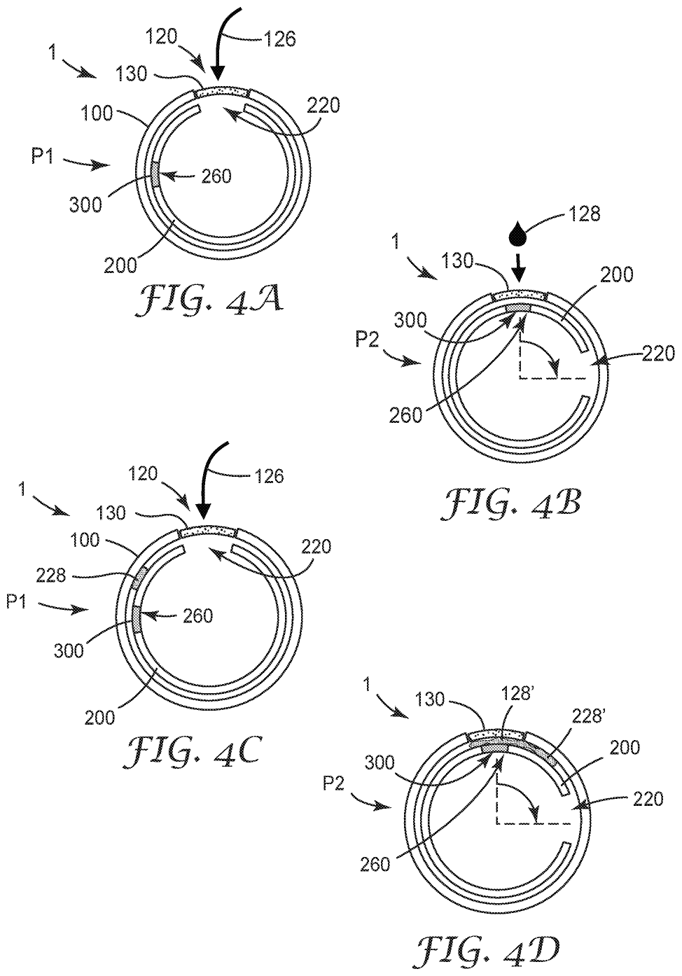

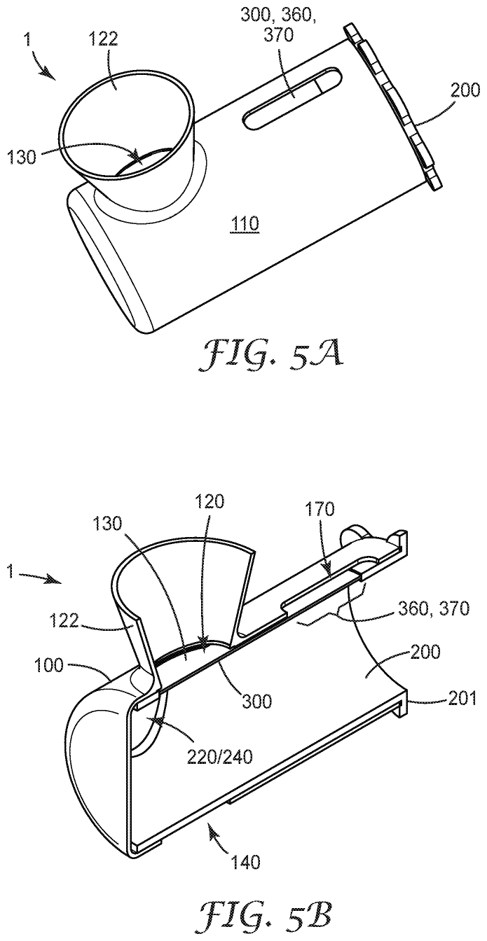

The embodiments shown in FIGS. 1A-1C and 6A-6C are generally used in a similar manner. The operation and positions are explained here with reference to the first embodiment (FIGS. 1A-1C) but they apply similarly to the second embodiment (FIGS. 6A-6C). The first part 100 and the second part 200 may have a first position P1 that is a sample collection position and a second position P2 that is a testing position. The first position P1 is shown, for example, in FIGS. 1A-1C and 4A, and the second position P2 is shown, for example, in FIGS. 4B and 5A-5B. In the first position P1, the first opening 120 and the porous sample collection media 130 of the first part 100 are aligned (e.g., registered) with the first opening 220 of the second part 200. The second opening 140 is also aligned (e.g., registered) with the second opening 240 of the second part.

In the first position P1, a user may exhale into the first opening 120 (e.g., into the mouthpiece 122). Exhalation airflow 126 (shown in FIG. 4A) passes through the thickness of the screen 132 (if included) and porous sample collection media 130. The porous sample collection media 130 at least partially occludes the first opening 120. The porous sample collection media 130 may have a major plane that is orthogonal to the direction of the exhalation airflow 126 passing through the thickness of the porous sample collection media 130. The exhalation airflow 126 leaves the housing 10 through the second openings 140, 240 and/or through the open end 150, 250. The porous sample collection media 130 captures viruses, pathogens, or other analytes, in the exhalation airflow 126 that passes through the porous sample collection media 130.

The first and second parts 100, 200 may be rotated to the second position P2, for example, by gripping the grip element 252 of the second part 200 and turning the second part 200 relative to the first part 100. The second part 200 may be turned until the sample receiving area 330 of the assay 300 is aligned with the porous sample collection media 130. The second part 200 may be constructed so that the first opening 220 and assay receptacle 260 are about 90 degrees apart. This means that the second part 200 is rotated by about 90 degrees to align the first opening 120 and porous sample collection media 130 with the assay receptacle 260 and the sample receiving area 330 of the assay 300. It will be understood by those skilled in the art that the first opening 220 and assay receptacle 260 may be positioned at different angles, for example separated by about 30 degrees to about 150 degrees, as long as in the first position P1 the first openings 120, 220 can be aligned without interference by the assay 300 and in the second position P2 the first opening 120 and the assay 300 can be aligned without interference by the first opening 220 or second opening 240 of the second part 200.

In the second position P2 the sample can be eluted by applying a liquid 128 to the porous sample collection media 130. The eluent passes through the porous sample collection media 130 onto the sample receiving area 330 of the assay 300. The eluent travels to the test area 360 of the assay 300 by wicking or capillary action, where the possible target virus, target pathogen, or other target analyte from the sample react with a testing reagent. The result, indicating either the presence or absence of the target virus, target pathogen, or other target analyte of interest, is displayed in a result display 370. The result display 370 may be viewed through the result viewing area 170 of the first part.

In some embodiments, the system 1, 2, may include a liquid reservoir 228 for providing a metered dose of liquid 128′. The liquid reservoir 228 may be attached to the first part 100 or the second part 200. The liquid reservoir 228 may be attached to accommodate applying the liquid (e.g., a metered dose) to the porous sample collection media 130. An exemplary embodiment of such a liquid reservoir 228 is shown in FIGS. 4C and 4D. In this embodiment, the liquid reservoir 228 is a liquid-filled blister attached to the second part 200 and disposed between the second part 200 and the first part 100. The liquid reservoir 228 may be ruptured when the first and second parts 100, 200 are rotated to the second position P2. The ruptured liquid reservoir 228′ releases the metered dose of liquid 128′, which elutes the sample from the porous sample collection media 130.

The sample collection system may further comprise a machine-readable optical label. Such labels may include, for example, a bar code and a QR (quick response) code. The machine-readable optical label may be configured to display the result of the assay. The machine-readable optical label may be used to read and record the result. An electronic reader capable of reading machine-readable optical labels may be used to read and record the result. An electronic reader may be, for example, a smart phone, a tablet, a laptop, or bar code reader or QR code reader. The electronic reader may further be used to transmit the result, for example, to a healthcare provider or to a database.

A method of using the sample collection system may include exhaling into the first opening (e.g., into the mouthpiece) to capture a sample in the porous sample collection media: moving (e.g., rotating) the first and second parts relative to one another to align the porous sample collection media with the assay (e.g., with the sample receiving area of the assay): applying a liquid to the porous sample collection media: and reading a result in the result display of the assay. The liquid may be applied in an amount suitable for eluting viruses, pathogens, or other analytes, captured in the porous sample collection media. A suitable amount of liquid may be determined as a ratio of liquid volume to the surface area of the porous sample collection media. For example, the volume of liquid may be in a range from 10 μm/cm2 to 400 μm/cm2, or from 10 μm/cm2 to 250 μm/cm2, or from 50 μm/cm2 to 150 μm/cm2. In some embodiments, the volume of liquid is from 50 μm to 500 μm. The method may further include reading the result display of the assay using an electronic reader.

The sample collection system may be provided as a kit. The kit may include the sample collection system as discussed above, and instructions for collecting a sample and testing the sample using the assay. The instructions may include instructions to: exhale along the airflow path to capture a sample in the porous sample collection media; move the first and second parts relative to one another to align the porous sample collection media with the assay; apply a liquid to the porous sample collection media; and read a result in a result display of the assay. The instructions may further include instructions to read a result display of the assay using an electronic reader.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A sample collection system comprising:
a housing comprising a first part and a second part, the first and second parts being movable relative to one another;
porous sample collection media disposed along an airflow path in the first part; and an assay disposed on the second part and constructed to receive a sample captured by the porous sample collection media.

2. The sample collection system of claim 1, wherein the airflow path comprises a through opening in the first part and the second part, and wherein the porous sample collection media occludes the through opening of the first part.

3. The sample collection system of claim 1, wherein the airflow path comprises a second through opening in each of the first and second parts.

4. The sample collection system of claim 1, wherein the first and second parts are rotatably movable relative to one another.

5. The sample collection system of claim 1, wherein the first part forms an outer tube and the second part forms an inner tube at least partially disposed inside the outer tube.

6. The sample collection system of claim 1, wherein the first part and the second part have a first position that is a sample collection position and a second position that is a testing position.

7. The sample collection system of claim 6, wherein in the first position, through holes extending through the first part and the second part are aligned.

8. The sample collection system of claim 7, wherein in the second position, the porous sample collection media is aligned with the assay.

9. The sample collection system of claim 6, wherein in the second position, the porous sample collection media is in contact with the assay.

10. The sample collection system of claim 6, wherein the assay comprises a sample receiving area, and wherein in the second position, the porous sample collection media is aligned with the sample receiving area.

11. The sample collection system of claim 6, wherein the assay comprises a sample receiving area, and wherein in the second position, the porous sample collection media is in contact with the sample receiving area.

12. The sample collection system of claim 1, wherein the first part comprises a transparent material in an area overlaying a result display of the assay in the second position.

13. The sample collection system of claim 1, wherein the first part comprises a screen disposed in the airflow path in front of the porous sample collection media.

14. The sample collection system of claim 13, wherein the first part further comprises a mouthpiece aligned with the porous sample collection media.

15. The sample collection system of claim 14, wherein the mouthpiece is removably coupled with the first part.

16. The sample collection system of claim 1, wherein the first part forms an outer tube having an outer surface with a non-circular cross section.

17. The sample collection system of claim 1, wherein the assay comprises a lateral flow assay or a vertical flow assay.

18. The sample collection system of claim 17, wherein the assay comprises a strip of material with a length, and wherein the length is parallel to a longitudinal axis of the second part.

19. The sample collection system of claim 17, wherein the assay comprises a strip of material with a length, and wherein the length is transverse to a longitudinal axis of the second part.

20. The sample collection system of claim 1, wherein the porous sample collection media comprises nonwoven material.

21-26. (canceled)
